SEQUENCE LISTING (2 sequences)

SEQ ID NO: 1 - protein, 488 aa, Ixodes scapularis
MSGLSLKLWIVAFFAFCLAEKEHGIVYPKMLESRAATGERMLKINDDLTLTLQKSKVFADDFLFSTTDGNEPIDYYTKAEDAERDIYHDSTHMASVRVTDDDGVEVEGILGERLRVKPLPAMARTSNGLRPHMLYEVDAHENGRPHDYGSTNTSNTPVESRTGSTKHSMYKIPLEIYPEVHLVVDSTFASAFKFDVYKVTRYFAVLTNAANLRYASFVFPKVQLRITGITMNKKPADEPYIHNVKGYEKYRNILFKETLEDFNKQMKSKPFYYTPDIVFLVTGKNMSEWVDGKLQHWVGGYAYLGTACSEWRVGMSEDPPTSYYGAYVFAHELAHNLGCQHDGDGASDWVKGHIGSADCPWDDGYLMSYKMQDERQYQFSYCCQREVRNLYNRPEFKCLRERYTTKTIKRSSKLPGRMTTLSNYCQRVYRYEKGMHADETYGVRDCKVKCTGNTNYWRLTVVDGTPCGKKKACILGKCVDDIKISKDD

SEQ ID NO: 2 - protein, 174 aa, Ixodes scapularis
MHQEGDFKMGHCSDLKVSALEIPYKGNKMSMVILLPEDVEGLSDLEEHLTAPKLLALLGGMYVTSDVNLHFPKFKLEQSMGLKDVLMAMGVKDFFTFLADLSGISATGNLCASDVIHKAFVEVNEEGTEAAAATAILMDCIPQVVNFFVDHPFMFLICSHDPDAVLFMGSIREL

(19) United States
(12) Patent Application Publication
(10) Pub. No.: US 2021/0030853 A1
(43) Pub. Date: Feb. 4, 2021

(54) TICK VACCINE

(71) Applicant: Technische Universität Braunschweig, Braunschweig (DE)

(72) Inventors: Michael HUST, Braunschweig (DE); Stefan DÜBEL, Braunschweig (DE)

(73) Assignee: Technische Universität Braunschweig, Braunschweig (DE)

(21) Appl. No.: 17/048,155

(22) PCT Filed: Apr. 17, 2019
(86) PCT No.: PCT/EP2019/059909

§ 371 Date: Oct. 16, 2020

(30) Foreign Application Priority Data
Apr. 17, 2018 (EP) 18167683.4

(51) Int. Cl. A61K 39/00; A61P 33/14

(52) U.S. Cl. CPC A61K 39/0003; A61P 33/14; A61K 2039/70; A61K 2039/55505; A61K 2039/575

(57) ABSTRACT

The present invention relates to combinations of tick-derived antigens for use as a tick vaccine.

The present invention relates to combinations of tick-derived antigens for use as a tick vaccine.

Ticks of the genus Ixodes are blood-feeding acarines infesting a large variety of vertebrates ranging from amphibians to mammals, including domestic animals and humans. They can be vectors for the transmission of diseases caused by different pathogens, including bacteria, viruses and parasites.

Borreliosis caused by bacteria of the genus Borrelia and tick-borne encephalitis (TBE) caused by the tick-borne encephalitis virus (TBEV) are among the most frequently occurring tick-borne diseases. In Europe, Northern America and Asia, borreliosis is mainly transmitted by ticks of the Ixodes genus (Shapiro 2014; Willyard 2014). In Germany only, about 60,000-200,000 annual cases of borreliosis have been reported (Hofmann et al., 2017). In the USA, the number of annual cases of borreliosis is estimated as being more than 300,000 (Nelson et al., 2015). In Europe, the number of TBE cases has strongly increased, in Germany only, the number has doubled between 2001 and 2006 (Frimmel et al., 2014).

In addition, ticks of the genus Ixodes are known to transmit diseases caused by further pathogens like the bacteria Francisella tularensis (tularemia/rabbit fever), Rickettsia spec. (e.g R. helvetica, Helvetica spotted fever), further Borrelia species (e.g. B. miyamotoi, relapsing fever illness) and Anaplasma phagocytophilum (human granulocytic ehrlichiosis), unicellular parasites such as Babesia Spp. (babesiosis) or viruses like Powassan virus (Choi et al., 2016; Eisen, 2018, Ismail and McBride, 2017; Vonesch et al., 2016).

Instead of developing separate vaccines against each individual pathogen, e.g. a new vaccine against borreliosis, the present inventors developed a vaccine against the vector, e.g. the tick itself.

Since the 1930s, it was observed that subjects frequently exposed to ticks may develop an immune response and resistance against ticks, i.e. acquired tick immunity. This may lead to reduced feeding, early disattachment from the host and even death of the tick (Gomes et al., 2015; Trager, 1939; Wikel, 1996). In the first 24 h after attachment, the tick has to overcome various host defense mechanisms such as blood coagulation, vasoconstriction, inflammatory processes as well as an immune response. Typically, during this time, no pathogens are transmitted. It was found that acquired tick immunity results in the reduction of the number of transmitted pathogens and thus to a reduced rate of infection (Dai et al., 2010; Nazario et al., 1998; Shattuck et al., 2014; Wikel, 1996).

Numerous attempts of using different immunogenic tick proteins as a candidate vaccine against ticks of various Ixodes species were reported: metalloproteases such as metalloproteases 1 or 2 (Metis1/Metis2) (Becker et al., 2015; Decrem et al., 2008), subolesin/akirin/4D8 (Contreras and de la Fuente, 2016; de la Fuente et al., 2013), CDK10 (Gomes et al., 2015), tick histamine release factor (Dai et al., 2010), aquaporin (Contreras and de la Fuente, 2017), anti-complement proteins (Daix et al., 2007; Valenzuela et al., 2000), and BM86 homologous proteins (Coumou et al., 2014). Further immunogenic proteins were identified by means of yeast display (Schuijt et al., 2011).

Several of these proteins were used in vaccine studies which, however, did not lead to the development of a clinically acceptable human vaccine.

Becker et al. (2015) have identified antibodies against several proteins from I. scapularis in human blood sera by M13 phage display. The sera were obtained from tick-bite sensitive donors who had been bitten multiple times, some of them reporting strong and instant responses such as itching or rashes. Among these proteins was a salivary gland metalloprotease 1 (complete CDS: AY_264367.1) which was indicated as the most promising candidate for vaccine development. Additional targets identified in tick-sensitive subjects included a conserved hypothetical protein (mRNA: XM_002402723.1), a putative solute carrier (mRNA: XM_002416261.1), a putative thrombin inhibitor (mRNA: XM_002408067.1), a putative ubiquitin protein ligase (mRNA: XM_002412768.1), a putative 60S ribosomal protein L14 (mRNA: XM_002403042.1), a putative E3 ubiquitin protein ligase Bre1 (mRNA: XM_002434300.1) and a putative DNA ligase (mRNA: XM_002406537.1), all from I. scapularis. No combination of two or more of these targets has been suggested. The immunogenic character of the thrombin inhibitor and other identified targets could not be confirmed.

In order to overcome the deficiency of prior art approaches, the present inventors provide a combination of several different antigens for the development of an efficient human vaccine. A first component of the vaccine is a metalloprotease 1 (MP1) protein from a tick of the genus Ixodes including immunogenic homologues and immunogenic fragments thereof. A second component of the vaccine is a thrombin inhibitor from a tick of the genus Ixodes including immunogenic homologues and immunogenic fragments thereof.

This combination has been developed by the present inventors from the identification of antibodies in blood sera of human subjects with acquired tick immunity. Here, antibodies against metalloprotease 1 and thrombin inhibitor from I. scapularis were identified by ORFeome phage display. From these results, the inventors concluded that a combination of these two components, optionally including further immunogenic polypeptides from an Ixodes organism, is useful as a tick vaccine.

Accordingly, a novel tick vaccine, e.g. a polypeptide- or nucleic acid-based tick vaccine is provided. The tick vaccine is for use in medicine, particularly for use in human medicine. Also encompassed is a use in veterinary medicine. The tick vaccine is directed against ticks, particularly against ticks of the genus Ixodes, more particularly against several different tick species of the genus Ixodes, e.g. at least one of I. ricinus, I. persulcatus, I. scapularis, and I. pacificus.

The tick vaccine of the present invention is capable of eliciting an enhanced humoral and/or cellular immune response compared to its individual components. Thus, an increased protection against ticks and concomitantly an increased efficacy against tick-borne diseases such as borreliosis may be achieved. The two components of the combination, namely metalloprotease 1 and thrombin inhibitor, are both present in tick saliva, but exhibit different functions, e.g. suppression of histamine secretion, proteolysis of blood clotting factors and inhibition of immune cells. By eliciting an immune response against both proteins a broad spectrum of physiological effects may be addressed leading to an increased protection.

Further, within Ixodes species, most saliva proteins a similar, but not completely identical. Immunization against a combination of several proteins leads to an improvement of cross species protection.

A first aspect of the invention relates to a combination comprising at least the following components:
(a) (i) a metalloprotease 1 from an Ixodes tick species particularly selected from I. ricinus, I. persulcatus, I. scapularis, and I. pacificus,
   (ii) a polypeptide having a sequence identity of at least 80%, 85%, 90%, 95% or 98% to a polypeptide according to (i),
   (iii) an immunogenic fragment of a polypeptide according to (i) or (ii) comprising at least 6, at least 8, at least 10, at least 12 or at least 15 contiguous amino acids of a polypeptide according to (i) or (ii), and/or (iv) a nucleic acid molecule encoding a polypeptide according to (i), (ii) or (iii), and
(b) (i) a thrombin inhibitor from an Ixodes tick species particularly selected from I. ricinus, I. persulcatus, I. scapularis, and I. pacificus,
   (ii) a polypeptide having a sequence identity of at least 80%, 85%, 90%, 95% or 98% of a protein according to (i),
   (iii) an immunogenic fragment of a polypeptide according to (i) or (ii) comprising at least 6, at least 8, at least 10, at least 12 or at least 15 contiguous amino acids of a polypeptide according to (i) or (ii), and/or
   (iv) a nucleic acid molecule encoding a polypeptide according to (i), (ii) or (iii).

In addition, the combination may comprise at least one further component selected from:
(c) (i) an immunogenic polypeptide (different from (a) and (b) as described above) from an Ixodes tick species particularly selected from I. ricinus, I. persulcatus, I. scapularis, and I. pacificus,
   (ii) a polypeptide having a sequence identity of at least 80%, 85%, 90%, 95% or 98% of a protein according to (i),
   (iii) an immunogenic fragment of a polypeptide according to (i) or (ii) comprising at least 6, at least 8, at least 10, at least 12 or at least 15 contiguous amino acids of a polypeptide according to (i) or (ii), and/or
   (iv) a nucleic acid molecule encoding a polypeptide according to (i), (ii) or (iii).

Component (a) (i) of the combination is a metalloprotease 1 from a tick species. This polypeptide is capable of proteolytical cleavage of fibrin(ogen) and inhibition of blood clotting (Decrem et al., 2008; Francischetti et al., 2003). MP1 is a member of the group of reprolysin-like metalloproteases which are involved in a manifold way in the modulation of the host immune system (Ali et al., 2014).

In a specific embodiment, component (a) (i) is a metalloprotease 1 from an Ixodes tick species selected from I. ricinus, I. persulcatus, I. scapularis, and I. pacificus. A preferred amino acid sequence of a metalloprotease 1 from I. scapularis is shown in SEQ ID NO: 1. A further preferred amino acid sequence of a metalloprotease 1 from I. scapularis is shown in UniProt B7PMT8.

The invention also encompasses an immunogenic homologue or an immunogenic fragment of the metalloprotease 1 (a) (i). An immunogenic homologue (a) (ii) may be selected from a polypeptide having a sequence identity of at least 80%, 85%, 90%, 95% or 98% of a polypeptide according to (a) (i).

An immunogenic fragment (a) (iii) may be selected from fragments of a polypeptide according to (i) or (ii) comprising at least 6, at least 8, at least 10, at least 12 or at least 15 contiguous amino acids of a polypeptide according to (a) (i) or (ii).

Further, the invention encompasses a nucleic acid molecule (a) (iv) encoding a polypeptide according to (a) (i), (ii) or (iii). The nucleic acid molecule may be a DNA or RNA which can be single-stranded or double-stranded. Preferably, the nucleic acid molecule is in operative linkage with an expression control sequence, e.g. a eukaryotic expression control sequence. The nucleic acid molecule may be present as such or located on a vector, e.g. a plasmid vector or a viral vector as known in the art.

Component (b) (i) of the combination is a thrombin inhibitor from a tick species. In a specific embodiment, component (b) (i) is a thrombin inhibitor A preferred amino acid sequence of a thrombin inhibtor from I. scapularis is shown in SEQ ID NO: 2 (UniProt B7PVJ0).

The invention also encompasses an immunogenic homologue or an immunogenic fragment of the thrombin inhibitor (b) (i). An immunogenic homologue (b) (ii) may be selected from a polypeptide having a sequence identity of at least 80%, 85%, 90%, 95% or 98% of a polypeptide according to (b) (i).

An immunogenic fragment (b) (iii) may be selected from fragments of a polypeptide according to (i) or (ii) comprising at least 6, at least 8, at least 10, at least 12 or at least 15 contiguous amino acids of a polypeptide according to (b) (i) or (ii).

Further, the invention encompasses a nucleic acid molecule (b) (iv) encoding a polypeptide according to (b) (i), (ii) or (iii). The nucleic acid molecule may be a DNA or RNA which can be single-stranded or double-stranded. Preferably, the nucleic acid molecule is in operative linkage with an expression control sequence, e.g. a eukaryotic expression control sequence. The nucleic acid molecule may be present as such or located on a vector, e.g. a plasmid vector or a viral vector as known in the art.

In addition to components (a) and (b), the combination of the invention may also comprise a component (c) as described above. Component (c) (i) may be an immunogenic tick polypeptide from an Ixodes tick species selected from I. ricinus, I. persulcatus, I. scapularis, and I. pacificus such as:

alternative complement activation inhibitor,

aquaporin,

BM86 (ATAQ, Ir86-2),

BM91,

CDK10,

cement protein RIM36,

Haa86,

HGag,

histamine-binding protein (HBP, 25 kDa salivary gland protein C, putative salivary lipocalin),

IL2 binding protein,

IRAC (anti-complement),

RmFER2 (ferritin 2),

Salp15 (SG-1, SG-2, SG-3, Salp15 like protein, 15 kDa salivary gland protein),

Salp25 (25 kDa salivary gland protein B, Lir4),

a serpin,

subolesin,

tick histamin release factor (THRF),

TSLPI (salivary lectin pathway inhibitor) and

64TRP (cement protein).

Components (c) (ii) or (iii) may be selected from immunogenic homologues or immunogenic fragments of component (c) (i).

Further, the invention encompasses a nucleic acid molecule (c) (iv) encoding a polypeptide according to (c) (i), (ii) or (iii). The nucleic acid molecule may be a DNA or RNA which can be single-stranded or double-stranded. Preferably, the nucleic acid molecule is in operative linkage with an expression control sequence, e.g. a eukaryotic expression control sequence. The nucleic acid molecule may be present as such or located on a vector, e.g. a plasmid vector or a viral vector as known in the art.

The polypeptide components (i), (ii) or (iii) may be provided as separate entities. These entities may comprise the polypeptides as such or fused or coupled to heterologous moieties. Alternatively, the polypeptide components (i), (ii) or (iii) may be provided as a single entity, comprising both components (a) and (b) and optionally further components, e.g. component (c). The single entity may be e.g. a genetic fusion polypeptide or a conjugate comprising a peptidic or non-peptidic moiety to which the polypeptide components are coupled.

The nucleic acid components (iv) may be provided as separate entities. These entities may comprise the nucleic acid molecules as such or fused or coupled to heterologous moieties. Alternatively, the nucleic acid components may be provided as a single entity, e.g. as a fused nucleic acid molecule encoding individual polypeptide components (i), (ii) or (iii) as separate entities or encoding a fusion polypeptide comprising both components (a) and (b) and optionally further components, e.g. component (c).

The combination of the present invention comprises at least two components (a) and (b) and optionally at least one further component (c) which may be immunogenic, i.e. causing an immune reaction including a humoral, i.e. antibody-based immune reaction and/or cellular, i.e. T-cell based immune reaction. Preferably, the term “immunogenic” according to the present invention is to be understood as causing an immune reaction in a vaccinated subject against a tick, particularly an Ixodes tick, more preferably resulting in an acquired tick immunity in a human subject. The acquired tick immunity may be determined by developing an antibody titer against tick proteins, particularly against the vaccinated tick antigens. The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Nucleic acid molecule” generally refers to any polynucleotide, which may be unmodified RNA or DNA or modified RNA or DNA. “Nucleic acid molecules” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions.

“Homologue” as the term is used herein, is a polynucleotide or polypeptide that differs from a reference polynucleotide or polypeptide respectively, but retains essential properties. A typical homologue of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical homologue of a polypeptide differs in amino acid sequence from another reference polypeptide. Generally, differences are limited so that the sequences of the reference polypeptide and the homologue are closely similar overall and, in many regions, identical. A homologue and reference polypeptide may differ in amino acid sequence by one or more substitutions (preferably conservative), additions and deletions in any combination. A substituted or inserted amino acid residue may or may not be one encoded by the genetic code.

“Identity” or “sequence identity” is a measure of the identity of nucleotide sequences or amino acid sequences. In general, the sequences are aligned so that the highest order match is obtained. “Identity” per se has an art-recognized meaning and can be calculated using published techniques.

The combination of the present invention may be provided as a composition comprising several components in a single container or a kit comprising individual components in separate containers.

The combination, or vaccine, according the invention, can be used to manufacture a medicament to produce antibody and/or T-cell immune response against a tick, particularly an Ixodes tick to protect a mammal, e.g. a human subject from tick-borne diseases. Preferably, the vaccine is for use in preventing an infection by a pathogen transmitted by a tick, e.g. a pathogen selected from Babesia spec., Borrelia spec., Francisella spec., Rickettsia spec., Anaplasma spec., Ehrlichia spec., tick-borne encephalitis virus (TBEV), Powassan virus, Colorado tick fever virus, Crimean-Congo hemorrhagic fever virus or Heartland virus.

Thus, the invention also relates to method for preventing a disease affecting a mammal, particularly to prevent or cure the transmission of pathogens by a tick, especially by an Ixodes tick, or the symptoms of diseases induced by a tick or pathogens transmitted by a tick, said method comprising the step of administrating to said mammal a sufficient amount of the vaccine according to the invention.

The combination or vaccine may comprise the active components of the invention in addition with one or more carriers and/or one or more adjuvants optionally together with further excipients such as anti-oxidants, buffers, preservatives.

Suitable carriers for administration of vaccines are well known in the art and can include buffers, gels, microparticles, implantable solids, solvents or any other agents by which the antigen of the vaccine can be introduced into a subject and be made sufficiently available to produce an immune response to the antigen.

Specific examples of carrier molecules for nucleic acid molecules are viral or non-viral vectors comprising the polynucleotide sequence according to the invention for a transfection or transformation of a target cell. Further carriers for nucleic acid molecules include lipid vesicles such as liposomes, particularly cationic lipid vesicles. Specific carriers for polypeptides include moieties to which polypeptides may be covalently coupled such as albumin or hemocyanine for improving their antigenic and immunogenic properties.

The term “adjuvant” has its usual meaning in the art of vaccine technology, i.e. a substance or a composition of matter which is not in itself capable of causing a specific immune response against the antigen of the vaccine, but which is nevertheless capable of enhancing the immune response against the antigen. In other words, the combination of vaccination with antigen and adjuvant induces an immune response against the antigen which is stronger than that induced by the antigen alone.

Examples of adjuvant molecules are saponine or suitable fractions thereof and lipopolysaccharides.

Other examples of adjuvants are metal salts, oil in water emulsions, lipids and/or derivatives thereof, aminoalkyl glucosaminide phosphate, immune stimulatory oligonucleotides QS21 or combinations thereof possibly in association with liposomes.

An adjuvant may comprise one or more carrier molecule(s), such as metal salt particles such as aluminum phosphate, aluminum hydroxide, calcium phosphate, magnesium phosphate, iron phosphate, calcium carbonate, magnesium carbonate, calcium sulphate, magnesium hydroxide or a double salt like ammonium-iron phosphate, potassium iron phosphate, calcium iron phosphate, calcium magnesium carbonate or a mixture of these salts or porous polymeric particles. Further suitable adjuvants block copolymers, ethylene copolymers, acrylic acid copolymers, mineral oil emulsions, squalene or mixtures thereof.

A further adjuvant is an immune stimulatory CpG oligonucleotide, preferably a CpG oligonucleotide having a length between 15 and 45 nucleotides.

The pharmaceutical combination or vaccine may also comprise other compounds which are used for enhancing the antigenicity or immunogenicity of active compounds by addition of immune modulators such as a cytokines, interferons, tumor necrosis factors, transforming growth factors, or colony stimulating factors preferably interleukin-2.

The pharmaceutical combination or vaccine of the invention may be in any suitable pharmaceutical form. Suitable solid or liquid pharmaceutical forms are, for example, granules, powders, pill, tablets, capsules, suppositories, syrups, emulsions, suspensions, creams, aerosols, drops or injectable solution in ampoule form, in whose preparation excipients and additives such as disintegrants, binders, coating agents, swelling agents, lubricants, flavorings, sweeteners or solubilizers are customarily used. In the particular case of a slow-release composition, the pharmaceutical composition may comprise a biocompatible matrix suitable for slow-release.

Regarding the pharmaceutical carrier, in general, the nature of the carrier will depend on the particular mode of administration being employed. For instance, parenteral formulations usually comprise injectable fluids that include pharmaceutically and physiologically acceptable fluids such as water, physiological saline, balanced salt solutions, or the like as a vehicle. For solid compositions, conventional non-toxic solid carriers can include, for example, pharmaceutical grades of mannitol, lactose, or magnesium stearate. In addition to biologically-neutral carriers, pharmaceutical compositions to be administered can contain minor amounts of non-toxic additives, such as wetting or emulsifying agents, preservatives, and pH buffering agents and the like.

The route of administration of the vaccine or pharmaceutical composition according to the present invention can be any suitable route of administration. It can be topical, intradermal, subcutaneous, oral, intravenous, parenteral, intra-peritoneal.

The vaccine is preferably administered orally or parenterally (including subcutaneous, intramuscular, intravenous, intradermal injection). Suitable parenteral administration formulations include aqueous and non-aqueous sterile injection solutions which may contain anti-oxidants, buffers, preservatives and solutes which render the formulation isotonic with the blood of the recipient, and aqueous and non-aqueous sterile suspensions which may include suspending agents or thickening agents.

Amounts and regimens for the administration of the vaccine or the pharmaceutical combination according to the present invention can be determined by those with ordinary skill in the clinical art.

The formulations may be presented, for example, in unit-dose or multi-dose containers, sealed ampoules and vials and may be stored in a freeze-dried condition requiring only the addition of the sterile liquid carrier immediately prior to use.

The vaccine formulation may also include adjuvant systems for enhancing the immunogenicity to the formulation, such as oil-in water systems and other systems known in the art. The dosage will depend on the specific activity of the vaccine and can be readily determined by routine experimentation.

### FIGURE LEGENDS

FIG. 1: Enhanced antigen-specific systemic humoral immune responses.

Serum obtained from each single mouse collected 14 days after the last immunization were analyzed for anti-thrombin inhibitor IgG, IgG1 and IgG2c antibodies by ELISA. Results are displayed as floating bars with indicating the mean per each group. (A) IgG and B (IgG1 and IgG2a).

FIG. 2: Increased cellular immune responses displayed by an enhanced number of antigen-specific cytokine producing cells. The quantity of cytokine producing cells were determined by ELISpot. Single splenocyte suspensions isolated 14 days after the last immunization were pooled for each group and re-stimulated with the TI protein. Results are expressed as spot forming units of 10⁶ unstimulated and re-stimulated cells. Results are displayed as box and whiskers plots for (A) IFNγ, (B) IL-2, (C) IL-4, and (D) IL-17.

FIG. 3: Generation of antigen-specific multifunctional CD4⁺ T cells. The quantity of cytokine producing CD4⁺ T cells was determined by a multiparametric flow cytometry approach. Spleens obtained 14 days after the last immunization were isolated and prepared from each single mouse and re-stimulated with the TI protein. Results are expressed as frequencies of CD4⁺ T cells producing the indicated cytokines and displayed as box and whiskers plots for (A) IFNγ, (B) TNF-α, (C) IL-4, and (D) IL-17.

FIG. 4: Enhanced antigen-specific systemic humoral immune responses. Serum obtained from each single mouse collected 7 days after the last immunization were analyzed for anti-MP1 IgG, IgG1 and IgG2c antibodies by ELISA. Results are displayed as floating bars with indicating the mean per each group. (A) IgG and B (IgG1 and IgG2c).

FIG. 5: Increased cellular immune responses displayed by enhanced number of antigen-specific cytokine producing cells. The quantity of cytokine producing cells was determined by ELISpot. Single splenocyte suspensions isolated 7 days after the last immunization were pooled for each group and re-stimulated with the MP1 protein. Results are expressed as spot forming units of 10⁶ unstimulated and re-stimulated cells. Results are displayed as box and whiskers plots for (A) IFNγ, (B) IL-2, (C) IL-4, and (D) IL-17.

FIG. 6: Generation of antigen-specific multifunctional CD4+ T cells. The quantity of cytokine producing CD4+ T cells was determined by a multiparametric flow cytometry approach. Spleens derived 7 days after the last immunization were isolated and prepared from each single mouse and re-stimulated with the MP1 protein. Results are expressed as frequencies of CD4^{÷} T cells producing the indicated cytokines and displayed as box and whiskers plots for (A) IFNγ, (B) TNF-α, (C) IL-4, and (D) IL-17.

FIG. 7: Enhanced antigen-specific systemic humoral immune responses. Serum obtained from each single mouse collected 7 days after the last immunization were analyzed for anti-TI and anti-MP1 IgG, IgG1 and IgG2c antibodies by ELISA. Results are displayed as floating bars with indicating the mean per each group. (A), (B) IgG and (C), (D) IgG1 and IgG2c.

FIG. 8: Changes in IgG1/IgG2c ratio following a combined antigen vaccination approach. Serum obtained from each single mouse collected 7 days after the last immunization were analyzed for anti-TI IgG1 and IgG2c antibodies. Results are displayed as floating bars with indicating the mean per each group. Displayed is the ratio of TI-specific IgG1/IgG2c.

### EXAMPLES

### Example 1: Immunization with the Thrombin Inhibitor from I. scapularis

A recombinant thrombin inhibitor (TI) produced in E. coli was tested as an antigen to be implemented in anti-tick vaccine formulations. For this purpose, an immunization study in mice was conducted.

### 1.1 Immunization Protocol

Female C57BL/6 mice (n=5/group) 8-12 weeks of age were immunized 3 times (prime-2-boost strategy) at day 0, 14, and 28 by intramuscular (i.m.) route. Each animal received a total dose of 100 μl containing 25 μg or 50 μg of the protein TI alone or co-administered with 50 μl (1:1 v/v) of either complete Freund's adjuvant (CFA) (1^{st} immunization) or incomplete Freund's adjuvant (IFA) (2^{nd} and 3^{rd} immunization) or alum hydroxide. Control animals received 100 μl PBS. Fourteen days after the last immunization, blood was collected for the analysis of antigen-specific serum antibodies and spleens were removed and processed to address cellular immune responses. Details are shown in Table 1.

TABLE 1

| | | | | Protein + | Total |
|---|---|---|---|---|---|
| | Group | Animals | | Adjuvants/Animal | amount |
| 1 | Control (PBS) | 5 | i.m. C57BL/6 | —/— | 100 μl |
| 2 | TI 25 μg | 5 | i.m. C57BL/6 | 25 μg/— | 100 μl |
| 3 | TI 50 μg | 5 | i.m. C57BL/6 | 50 μg/— | 100 μl |
| 4 | TI 25 μg + | 5 | i.m. C57BL/6 | 25 μg/50 μl | 100 μl |
| | CFA and IFA** | | | | |
| 5 | TI 50 μg + | 5 | i.m. C57BL/6 | 50 μg/50 μl | 100 μl |
| | CFA and IFA** | | | | |
| 6 | TI 25 μg + Alum | 5 | i.m. C57BL/6 | 25 μg/50 μl | 100 μl |
| 7 | TI 50 μg + Alum | 5 | i.m. C57BL/6 | 50 μg/50 μl | 100 μl |

### 1.2 Detection of Antigen-Specific Humoral Immune Responses

An Enzyme-linked Immunosorbent Assay (ELISA) was conducted to investigate the induction of IgG antibodies and the corresponding isotypes (IgG1 and IgG2c) against the TI. For this, high binding protein plates were coated with the protein (2 μg/ml in 0.05 M carbonate buffer) and incubated over night at 4° C. On the next day, the plates were washed und blocked for 2 h with 3% BSA (bovine serum albumin) in PBS to prevent unspecific binding. Following, 2-fold serial dilutions (starting with 1:1000) of the serum samples were incubated in 3% BSA/PBS for 2 h at 37° C. After washing with 1% BSA/PBS/0.05% Tween the plates were incubated for 1 h with the biotinylated goat-anti mouse IgG, or IgG1 or IgG2c antibodies. Then, the plates were washed again und incubated with for 30 min with peroxidase-conjugated streptavidin. Finally, the reaction was developed by adding ABTS [2,20-azino-bis(3-ethylbenzthiazoline-6-sulfonic acid)] in 0.1 M citrate-phosphate buffer containing 0.01 H₂O₂ and measured after 5 min at an OD (optical density) of 405 nm. Endpoint titers are expressed as absolute values of the last dilution giving an OD405 nm being 2 times higher than the negative control (Blank).

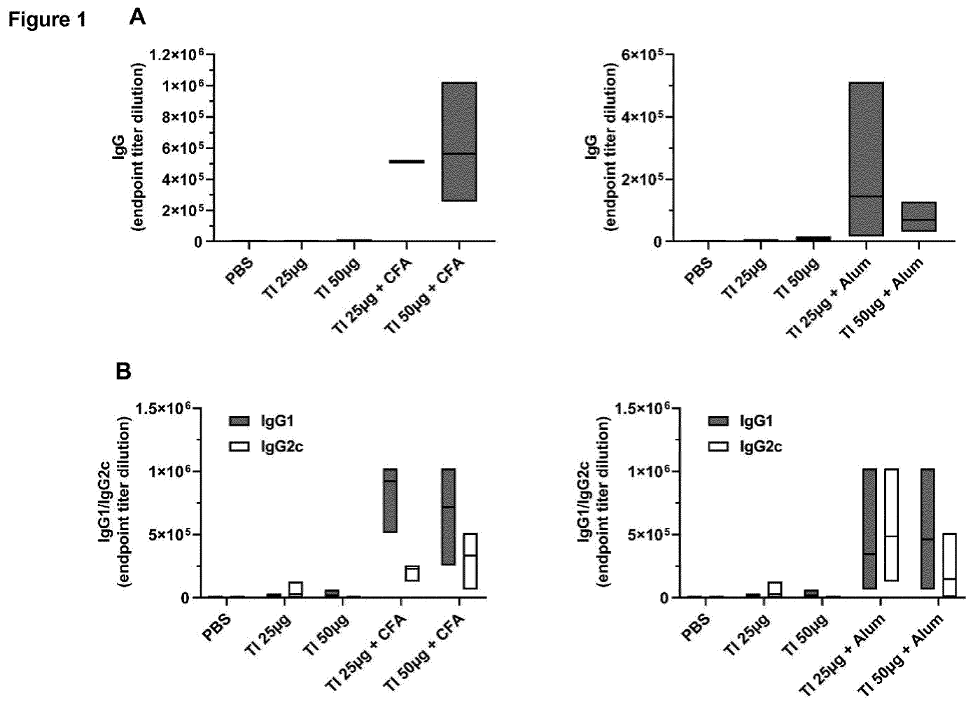

The obtained results revealed the induction of TI-specific humoral immune response following the prime-2-boost immunization strategy. More detailed, elevated anti-TI IgG titers were detected in all groups immunized with the protein co-administered with adjuvant as compared to antigen alone or control group (FIG. 1A). The further analysis of the IgG isotypes, IgG1 and IgG2c showed enhanced titers in all mice which received the antigen combined with one of the adjuvants with a slightly higher titer observed for IgG1 as compared to IgG2c (FIG. 1B). Comparing the two different antigen concentrations of 25 μg and 50 μg used for the immunization study, no major differences were observed for the induction of anti-TI titers IgG, IgG1 and IgG2c titers indicating that already lower amounts of the antigen are sufficient to induce strong humoral immune responses. Since the distribution of IgG1 and IgG2c provides indication for T-helper (Th) cell polarization, the data suggest rather the stimulation of Th-2 than Th-1 cells.

### 1.3 Detection of Antigen-Specific Cellular Immune Responses

To gain insight into the quantity of TI-specific induction of cytokine secreting cells (IFNγ, IL-2, IL-4, IL-17) an Enzyme-linked Immuno Spot Assay (ELISpot) was performed. To this end, plates with hydrophobic high protein binding Immobilon-P-Membrane were coated with anti-IFNγ, anti-IL-2, anti-IL-4, and anti-IL-17 antibodies diluted in PBS and incubated over night at 4° C. Unspecific binding sites were blocked for 2 h at room temperature with 200 μl/well of complete RPMI medium. Then, 5*10⁵ splenocytes/well were added and incubated in the absence (unstimulated) or presence of the TI protein (5 μg/ml). As positive control served splenocytes stimulated with the mitogen concanavalin A (5 μg/ml). The cells were incubated for 18 h (IFNγ) or 48 h (IL-2, IL-4, IL-17) at 37° C. Afterwards, the plates were washed with 0.01% Tween/PBS and incubated for further 2 h in the presence of the corresponding biotinylated detection antibodies. After another wash step, the cells were incubated with a peroxidase-conjugated streptavidin for 1 h at room temperature. Following, the plates were washed again and the cytokine secreting cells were detected by adding AEC substrate diluted in 0.1 M acetate buffer supplemented with 0.05% H₂O₂. The reaction was stopped depending on the development of the spots by adding distilled water. The samples were analyzed using the ImmunoSpot Image Analyzer software (CTL-Europe GmbH). Results are expressed as Spot-forming units calculated for 1*10⁶ cells.

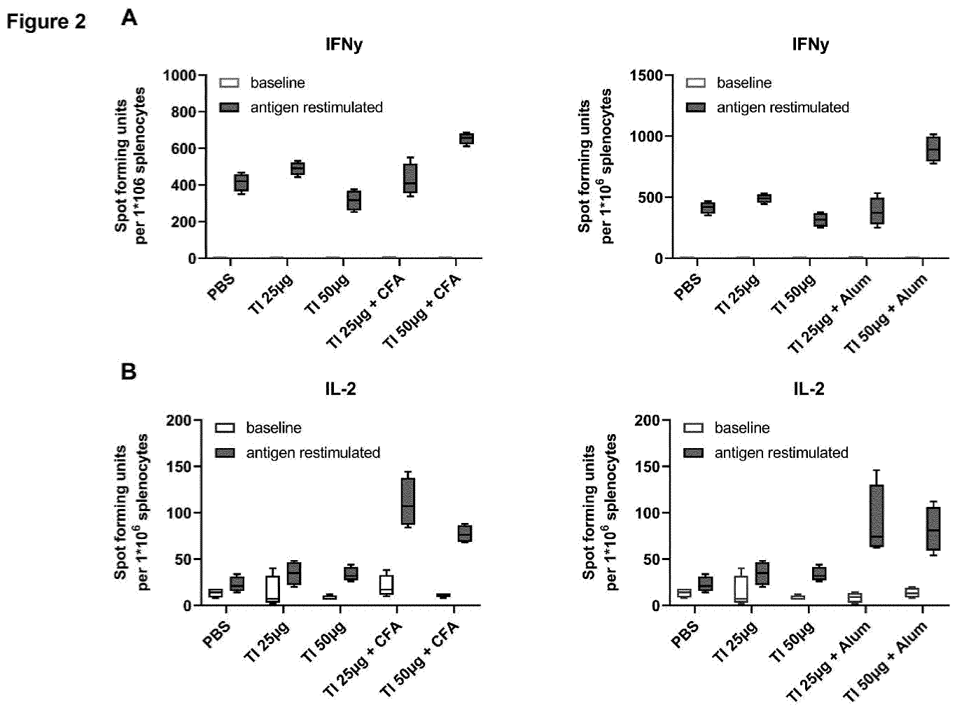

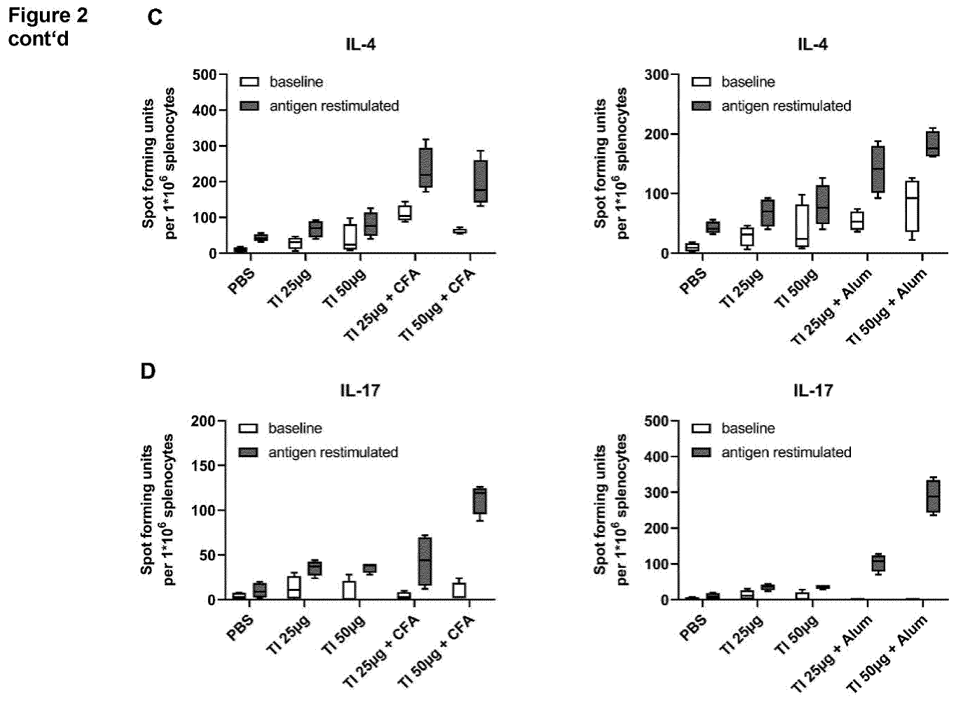

In vitro stimulation of splenocytes with the antigen displayed a strong increase in the number of IFNγ, IL-2, IL-4, and IL-17-secreting cells in the groups which were vaccinated with the TI protein co-administered with adjuvant as compared to unstimulated cells and the groups which received the antigen alone or PBS (FIG. 2A-D). Since IFNγ, IL-2, IL-4, and IL-17-secreting cells are considered as an indicator for Th-1, Th-2 or Th-17 biased immunity, the obtained results suggest the generation of a mixed/balanced Th-cell responses. Interestingly, the elevated number of IL-2 and IL-4 secreting splenocytes were found to be independent of the used antigen concentration, whereas the number of IFNγ and IL-17 secreting cells were mainly enhanced in the groups which were immunized with the higher amount of TI protein. Thus, the amount of used protein might impact Th-cell polarization.

Next, a multiparametric flow cytometry approach was applied to address the occurrence and functionality of antigen-specific CD4 and CD8 T cells. Single splenocyte suspension with 5*10⁶ cells/well were incubated with either medium alone or with TI (20 μg/ml) at 37° C. for 4 h. Afterwards brefeldin (5 μg/ml) and monensin (3 μg/ml) were added to the cells to prevent cytokine secretion or receptor internalization, respectively, thereby allowing cytokine accumulation inside the cells. Then, the cells were incubated for further 12 h followed by a surface marker (CD3, CD4, CD8, dead cell marker) and intracellular cytokine staining (IFNγ, IL-2, IL-4, TNFα and IL-17). To this end, splenocytes were incubated with the appropriate antibody cocktail diluted in PBS including the dead cell marker for 20 min at 4° C. After washing with PBS, cells were incubated for 30 min with a fixation/permeabilization buffer followed by the intracellular cytokine staining for 20 min. The cells were acquired using the BD LSRFortessa flow cytometry and analyzed using the FlowJo V10 software. Viable singlet lymphocytes were gated for CD3⁺CD4⁺ or CD3⁺CD8⁺ and analyzed for the frequency of IFNγ, IL-2, IL-4, TNFα and IL-17 producing T cells.

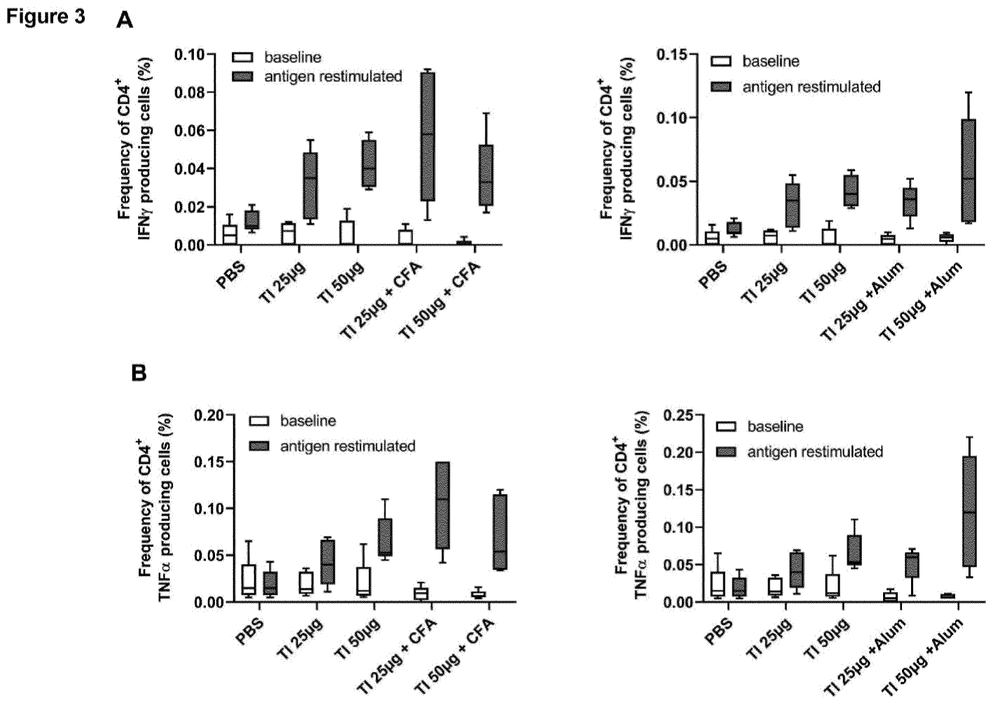

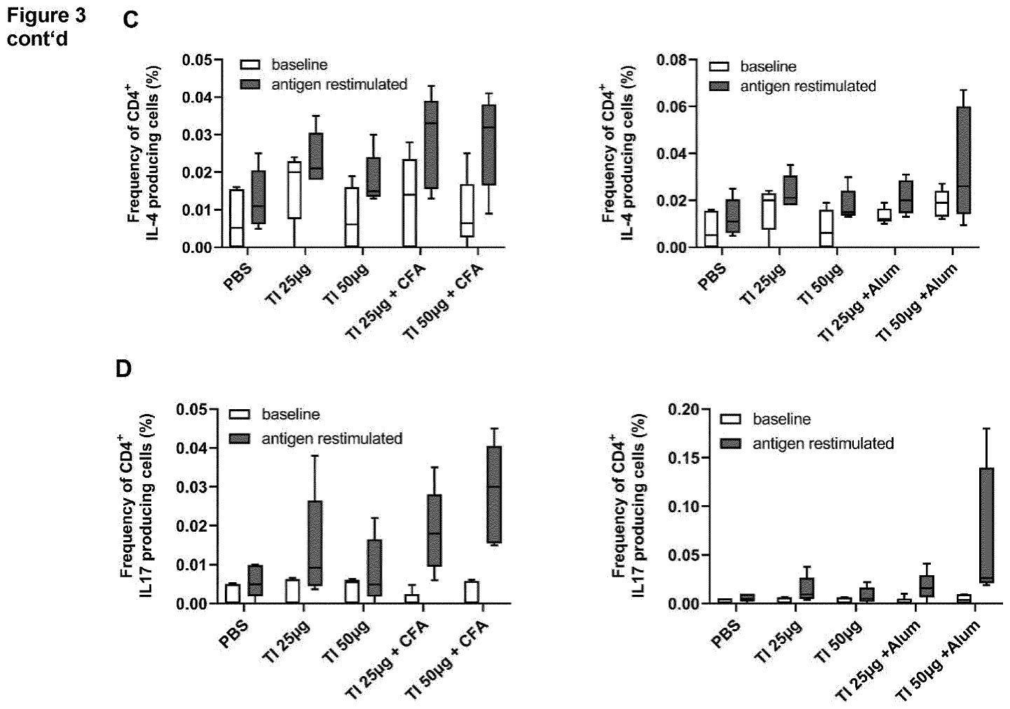

The analysis of multifunctional T cells characterized by the simultaneous production of different cytokines following re-stimulation revealed an enhanced frequency of IFNγ producing CD4⁺ T cells isolated from mice immunized with the TI protein alone as compared to the control group (FIG. 3A). The supplementation of the vaccine formulation with complete/incomplete Freund's adjuvant or alum resulted only in a marginal elevated level of IFNγ producing CD4⁺ T cells. However, adjuvantation could boost to some extend the frequencies of TNFα and IL-17 producing CD4⁺ T cells, whereas the frequency of IL-4 secreting CD4⁺ T cells were not affected (FIG. 3B-D). The analysis of multifunctional CD8⁺ T cell did not reveal changes in the frequencies of IFNγ and TNFα producing cells. The obtained data confirm the presence of TI-specific multifunctional CD4⁺ T cells following immunization and identify CD4⁺ T cells as one crucial cytokine-secreting immune cell population observed in FIG. 2.

In conclusion, the conducted studies demonstrate the generation of TI-specific IgG antibodies and their corresponding isotypes, thereby showing a clear seroconversion following immunization with the vaccine antigen candidate. The obtained data clearly show that immunization with the TI protein not only generates antigen-specific humoral responses, but also activates antigen-specific cellular immunity, especially multifunctional CD4⁺ T cells. The dose of antigen as well as the choice of adjuvant can affect/boost the immune response in diverse directions. The studies convincingly depict that the TI protein represents a suitable protein for an anti-tick vaccine.

### Example 2: Immunization with Metalloprotease 1 from I. scapularis

The metalloprotease (MP1) represents a salivary gland protein found to be secreted by the tick specie Ixodes scapularis. A recombinant MP1 produced in E. coli was tested as an antigen to be implemented in anti-tick vaccine formulations. For this purpose, an immunization study in mice was conducted.

### 2.1 Immunization Protocol

Female C57BL/6 mice (n=5/group) 8-12 weeks of age were immunized 3 times (prime-2-boost strategy) at day 0, 7, and 21 by intramuscular (i.m.) route. Each animal received a total dose of 100 μl containing 25 μg of the protein MP1 alone or co-administered with 50 μl (1:1 v/v) of either complete (1^{st} immunization) or incomplete Freund's adjuvant (**2^{nd} and **3^{rd} immunization). Since MP1 was solved in 2 M urea, which was diluted to 0.5 M in the vaccine formulation, control animals received 100 μl of 0.5 M Urea in PBS. Another control group were only injected with CFA/IFA. Seven days after the last immunization, blood was collected for the analysis of antigen-specific serum antibodies and spleens were removed and processed to address cellular immune responses. Details are shown in Table 2.

TABLE 2

| | | | | Protein + | Total |
|---|---|---|---|---|---|
| | Group | Animals | | Adjuvants/Animal | amount |
| 1 | 0.5M Urea in | 5 | i.m. C57BL/6 | —/— | 100 μl |
| | PBS | | | | |
| 2 | MP1 25 μg | 5 | i.m. C57BL/6 | 25 μg/— | 100 μl |
| 3 | MP1 25 μg + | 5 | i.m. C57BL/6 | 25 μg/50 μl | 100 μl |
| | CFA and IFA** | | | | |
| 4 | CFA/IFA** alone | 5 | i.m. C57BL/6 | —/50 μl | 100 μl |

### 2.2 Detection of Antigen-Specific Humoral Immune Responses

An Enzyme-linked Immunosorbent Assay (ELISA) was conducted to investigate the induction of IgG antibodies and the corresponding isotypes (IgG1 and IgG2c) against the MP1 antigen. For this, high binding protein plates were coated with the protein (2 μg/ml in 0.05 M carbonate buffer) and incubated over night at 4° C. On the next day, the plates were washed und blocked for 2 h with 3% BSA (bovine serum albumin) in PBS to prevent unspecific binding. Following, 2-fold serial dilutions (starting with 1:1000) of the serum samples were incubated in 3% BSA/PBS for 2 h at 37° C. After washing with 1% BSA/PBS/0.05% Tween the plates were incubated for 1 h with the biotinylated goat-anti mouse IgG, or IgG1 or IgG2c antibodies. Then, the plates were washed again und incubated with for 30 min with peroxidase-conjugated streptavidin. Finally, the reaction was developed by adding ABTS [2,20-azino-bis(3-ethylbenzthiazoline-6-sulfonic acid)] in 0.1 M citrate-phosphate buffer containing 0.01 H₂O₂ and measured after 5 min at an OD (optical density) of 405 nm. Antigen-specific endpoint titers are expressed as absolute values of the last dilution giving an OD405 nm being 2 times higher than the negative control (Blank).

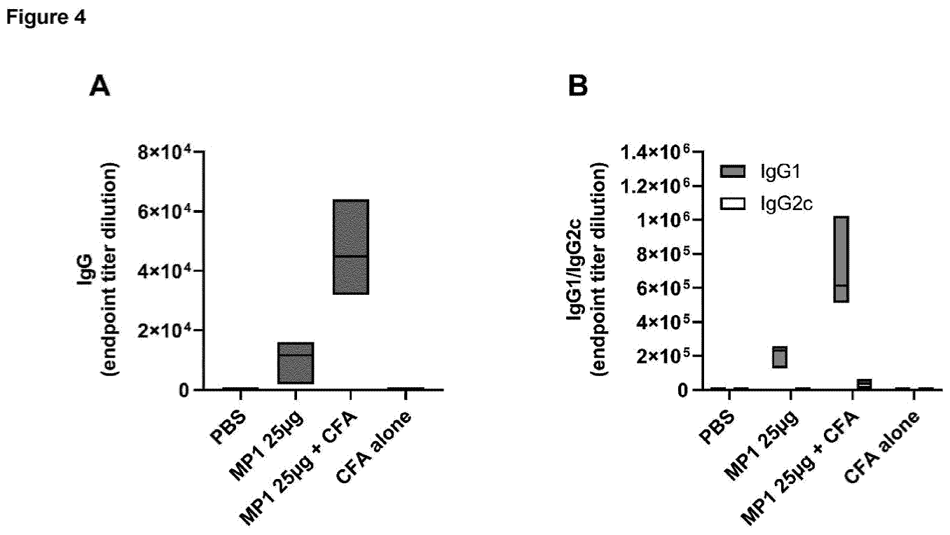

The obtained results revealed the generation of MP1-specific humoral immune responses following a prime-2-boost immunization strategy. More detailed, elevated anti-MP1 IgG titers were detected in all groups immunized with the protein alone or co-administered with adjuvant as compared to the control groups (FIG. 4A). The further analysis of the IgG isotypes, IgG1 and IgG2c showed mainly enhanced IgG1 titers in mice, which either received the antigen alone or combined with CFA/IFA, whereas only a minor increase of IgG2c was observed in general (FIG. 4B). Although mice that were administered with the protein MP1 alone displayed already antigen-specific IgG and IgG1 titer, the amount of antibody titers could be strongly increased by adding the CFA/IFA to the vaccine formulation. Furthermore, the results demonstrate the activation of Th-2 cells but not Th-1 cells, as pointed by the ratio of IgG1 and IgG2c antibodies respectively.

### 2.3 Detection of Antigen-Specific Cellular Immune Responses

To gain insight into the quantity of MP1-specific induction of cytokine secreting cells (IFNγ, IL-2, IL-4, IL-17) an Enzyme-linked Immuno Spot Assay (ELISpot) was performed. To this end, plates with hydrophobic high protein binding Immobilon-P-Membrane were coated with anti-IFNγ, anti-IL-2, anti-IL-4, and anti-IL-17 antibodies diluted in PBS and incubated over night at 4° C. Unspecific binding sites were blocked for 2 h at room temperature with 200 μl/well of complete RPMI medium. Then, 5*10⁵ splenocytes/well were added and incubated in the absence (unstimulated) or presence of the MP1 protein (5 μg/ml). As positive control served splenocytes stimulated with the mitogen concanavalin A (5 μg/ml). The cells were incubated for 18 h (IFNγ) or 48 h (IL-2, IL-4, IL-17) at 37° C. Afterwards, the plates were washed with 0.01% Tween/PBS and incubated for further 2 h in the presence of the corresponding biotinylated detection antibodies. After another wash step, the cells were incubated with a peroxidase-conjugated streptavidin for 1 h at room temperature. Following, the plates were washed again and the cytokine secreting cells were detected by adding AEC substrate diluted in 0.1 M acetate buffer supplemented with 0.05% H₂O₂. The reaction was stopped depending on the development of the spots by adding distilled water. The samples were analyzed using the ImmunoSpot Image Analyzer software (CTL-Europe GmbH). Results are expressed as Spot-forming units calculated for 1*10⁶ cells.

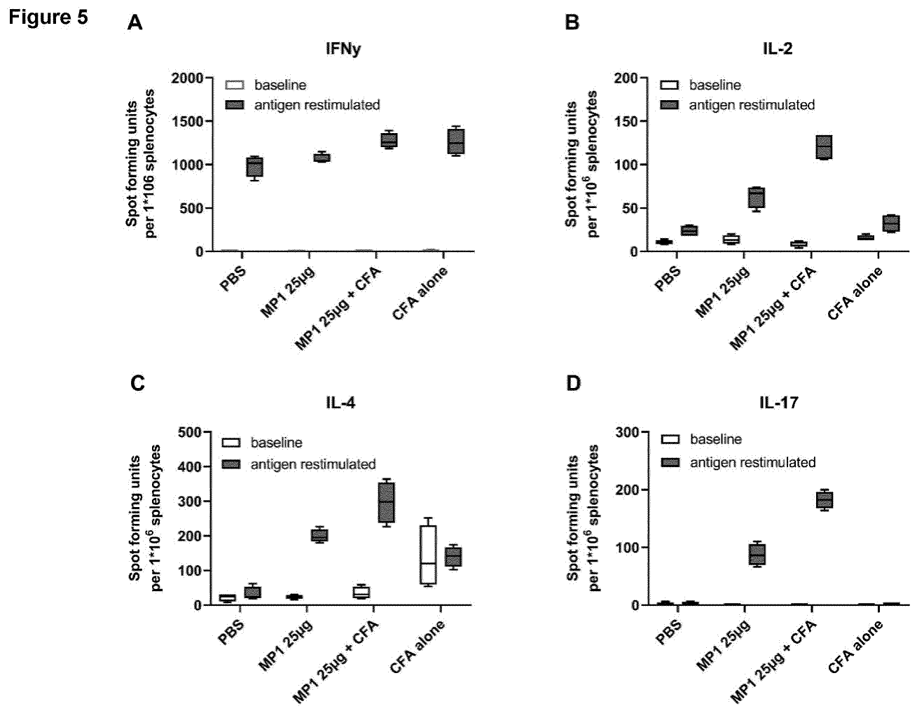

In vitro stimulation of splenocytes with MP1 antigen displayed an overall strong increase in the number of IFNγ, IL-2, IL-4, and IL-17-secreting cells. An elevated number of IFNγ secreting cells were observed for all MP1 restimulated splenocytes independent of the immunization status, thereby indicating an immune modulatory capacity of the protein itself on IFNγ producing cells. Nevertheless, mice, which received MP1 combined with CFA/IFA, displayed the highest level of IFNγ⁺ cells (FIG. 5A). The number of IL-2, IL-4 and IL-17 producing cells were mainly increased in the restimulated groups immunized with the antigen alone or together with the adjuvant as compared to the control groups. The addition of CFA to the antigen led to further enhanced number of cytokine secreting cells (FIG. 5B-D). The obtained results suggest the induction of a mixed Th-1/Th-2/Th-17 cell response.

Next, a multiparametric flow cytometry approach was applied to address the occurrence and functionality of antigen-specific CD4 and CD8 T cells. Single splenocyte suspension with 5*10⁶ cells/well were incubated with either medium alone or with MP1 (20 μg/ml) at 37° C. for 4 h. Afterwards brefeldin (5 μg/ml) and monensin (3 μg/ml) were added to the cells to prevent cytokine secretion or receptor internalization, respectively, thereby allowing cytokine accumulation inside the cells. Then, the cells were incubated for further 12 h followed by a surface marker (CD3, CD4, CD8, dead cell marker) and intracellular cytokine staining (IFNγ, IL-2, IL-4, TNFα and IL-17). To this end, splenocytes were incubated with the appropriate antibody cocktail diluted in PBS including the dead cell marker for 20 min at 4° C. After washing with PBS, cells were incubated for 30 min with a fixation/permeabilization buffer followed by the intracellular cytokine staining for 20 min. The cells were acquired using the BD LSRFortessa flow cytometry and analyzed using the FlowJo V10 software. Viable singlet lymphocytes were gated for CD3⁺CD4⁺ or CD3⁺CD8⁺ and analyzed for the frequency of IFNγ, IL-2, IL-4, TNFα and IL-17 producing T cells.

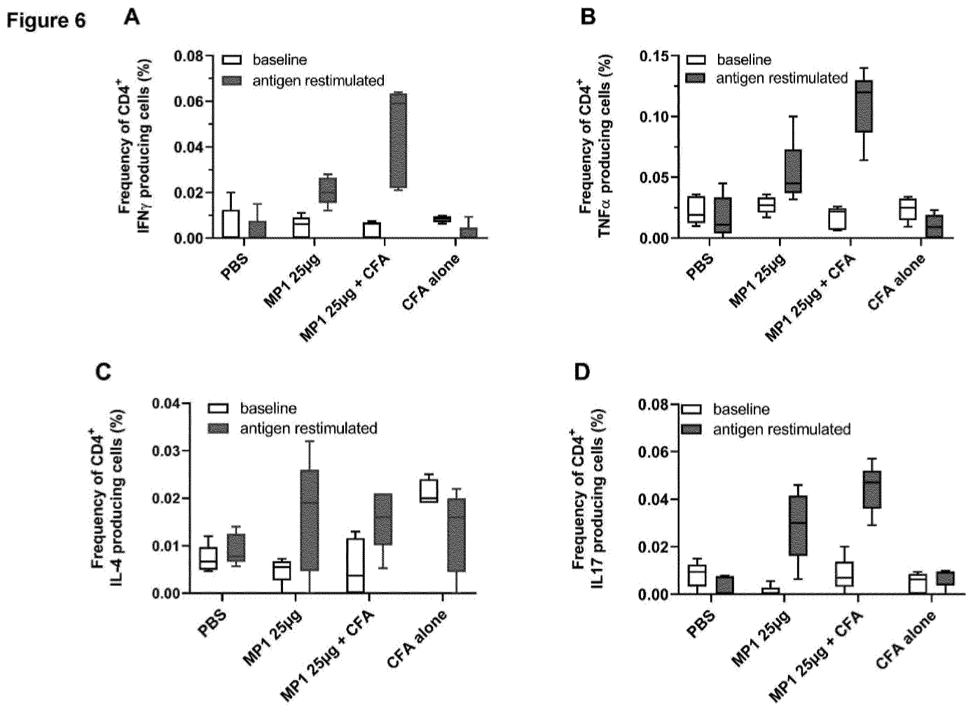

The analysis of multifunctional T cells characterized by the simultaneous production of different cytokines following re-stimulation revealed enhanced frequencies of IFNγ as well as TNFα producing CD4⁺ T cells isolated from mice immunized with the MP1 protein alone as compared to the control groups (FIG. 6A-B). The supplementation of the vaccine formulation with CFA/IFA could even further increase the level of IFNγ and TNF a producing CD4⁺ T cells. Furthermore, immunization with MP1 alone or in combination with CFA induced the activation of IL-17 secreting CD4 T cells, whereas IL-4 producing CD4 T cells were not detected (FIG. 6C-D). The analysis of multifunctional CD8⁺ T cell did not reveal changes in the frequencies of IFNγ and TNFα producing cells. However, the obtained data confirm the presence of MP1-specific multifunctional CD4⁺ T cells following immunization and identify CD4⁺ T cells as one crucial cytokine-secreting immune cell population observed in FIG. 5. In contrast to the data obtained above, the CD4 T cell analyses suggest rather the induction of a mixed Th-1/Th-17 immune response than a Th-2 one.

In conclusion, the conducted studies demonstrate the generation of MP1-specific IgG antibodies, especially of IgG1, thereby pointing a clear seroconversion following immunization with the vaccine antigen candidate. The obtained data clearly show that immunization with the MP1 protein not only generates antigen-specific humoral responses, but also activates antigen-specific cellular immunity, especially multifunctional CD4⁺ T cells. However, it needs to be considered that the high number of IFNγ-producing cells shown in FIG. 5A might be, to some extent, due to a slight contamination with bacterial components in course of the production process. Nevertheless, the analysis of the cytokine secreting CD4 T cells convincingly show the antigen-dependent reactivation of T cells. The performed studies strongly depict that the MP1 protein represents a suitable protein for an anti-tick vaccine.

### Example 3: Combined Antigen Approach

The already conducted immunization revealed a high immunogenic potential of the two proteins, thrombin inhibitor (TI) protein and the metalloprotease (MP1), secreted by the tick species Ixodes scapularis, when administered together with an adjuvant. To address whether the combination of these proteins influences antigen-specific immunity, immunization studies in mice were conducted.

### 3.1 Immunization Protocol

Female C57BL/6 mice (n=5/group) 8-12 weeks of age were immunized 3 times (prime-2-boost strategy) at day 0, 7, and 21 by intramuscular (i.m.) route. Each animal received a total dose of 100 μl containing 25 μg of the protein MP1 alone or co-administered with 50 μl (1:1 v/v) of either complete (1^{st} immunization) or incomplete Freund's adjuvant (**2^{nd} and **3^{rd} immunization). Seven days after the last immunization, blood was collected for the analysis of antigen-specific serum antibodies. Details are shown in Table 3.

TABLE 3

| | | | | Protein + | Total |
|---|---|---|---|---|---|
| | Group | Animals | | Adjuvants/Animal | amount |
| 1 | TI 25 μg + MP1 | 5 | i.m. C57BL/6 | 25 μg + | 100 μl |
| | 25 μg + Alum | | | 25 μg/50 μl | |
| 2 | Alum alone | 5 | i.m. C57BL/6 | 50 μl | 100 μl |

### 3.2 Detection of Antigen-Specific Humoral Immune Responses

An Enzyme-linked Immunosorbent Assay (ELISA) was conducted to investigate the induction of IgG antibodies and the corresponding isotypes (IgG1 and IgG2c) against the TI and MP1 antigen. For this, high binding protein plates were coated with the protein (2 μg/ml in 0.05 M carbonate buffer) and incubated over night at 4° C. On the next day, the plates were washed and blocked for 2 h with 3% BSA (bovine serum albumin) in PBS to prevent unspecific binding. Following, 2-fold serial dilutions (starting with 1:1000) of the serum samples were incubated in 3% BSA/PBS for 2 h at 37° C. After washing with 1% BSA/PBS/0.05% Tween the plates were incubated for 1 h with the biotinylated goat-anti mouse IgG, or IgG1 or IgG2c antibodies. Then, the plates were washed again and incubated with for 30 min with peroxidase-conjugated streptavidin. Finally, the reaction was developed by adding ABTS [2,20-azino-bis(3-ethylbenzthiazoline-6-sulfonic acid)] in 0.1 M citrate-phosphate buffer containing 0.01 H₂O₂ and measured after 5 min at an OD (optical density) of 405 nm. Antigen-specific endpoint titers are expressed as absolute values of the last dilution giving an OD405 nm being 2 times higher than the negative control (Blank).

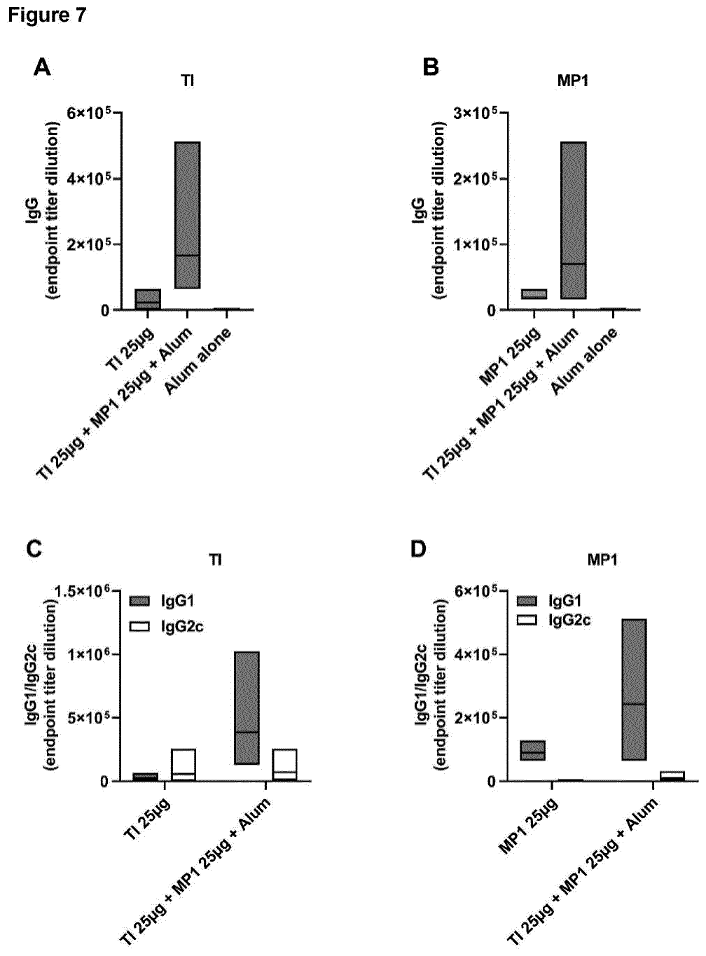

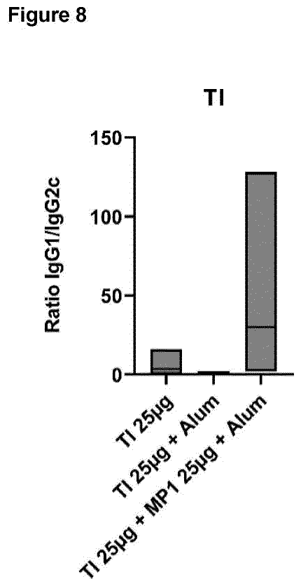

The obtained results revealed the generation of TI-specific as well as MP1-specific humoral immune responses following a prime-2-boost immunization strategy. Elevated anti-TI- and MP1 IgG titers were detected in all groups immunized with the protein co-administered with the adjuvant alum (FIG. 7A-B). The further analysis of the IgG isotypes, IgG1 and IgG2c showed mainly enhanced IgG1 titers whereas only a minor increase of IgG2c was observed in general (FIG. 7B-C). Compared to the immunization studies with the corresponding single antigen, no major differences in IgG titers were observed. However, a shift in the IgG1/IgG2c ratio was detected for the TI protein. Here, mice that were only vaccinated with TI and alum displayed a balanced distribution of IgG1 and IgG2c. In contrast, mice that were vaccinated with both antigens showed a strong reduced IgG2c response and a significant shift to IgG1 (FIG. 8).

The data suggest that the combination of antigens induces an equal strong humoral immune response as compared to single antigen immunization, but can influence the distribution of antibody subtypes. The changes in antibody balances will most probably also affect the generation of antigen-specific cellular immunity. These changes lead to a synergistic effect resulting in an overall improved vaccine response.

### LIST OF REFERENCES

Ali, A., Tirloni, L., Isezaki, M., Seixas, A., Konnai, S., Ohashi, K., da Silva Vaz Junior, I., Termignoni, C., 2014. Reprolysin metalloproteases from Ixodes persulcatus, Rhipicephalus sanguineus and Rhipicephalus microplus ticks. Exp. Appl. Acarol. 63, 559-578.
Becker, M., Felsberger, A., Frenzel, A., Shattuck, W. M. C., Dyer, M., Kugler, J., Zantow, J., Mather, T. N., Hust, M., 2015. Application of M13 phage display for identifying immunogenic proteins from tick (Ixodes scapularis) saliva. BMC Biotechnol. 15, 43.
Choi, E., Pyzocha, N. J., Maurer, D. M., 2016. Tick-Borne Illnesses. Curr Sports Med Rep 15, 98-104.
Connor, D. O., Zantow, J., Hust, M., Bier, F. F., von Nickisch-Rosenegk, M., 2016. Identification of Novel Immunogenic Proteins of Neisseria gonorrhoeae by Phage Display. PLoS ONE 11, e0148986.
Contreras, M., de la Fuente, J., 2017. Control of infestations by Ixodes ricinus tick larvae in rabbits vaccinated with aquaporin recombinant antigens. Vaccine 35, 1323-1328.
Contreras, M., de la Fuente, J., 2016. Control of Ixodes ricinus and Dermacentor reticulatus tick infestations in rabbits vaccinated with the Q38 Subolesin/Akirin chimera. Vaccine 34, 3010-3013. https://doi.org/10.1016/j.vaccine.2016.04.092
Coumou, J., Wagemakers, A., Trentelman, J. J., Nijhof, A. M., Hovius, J. W., 2014. Vaccination against Bm86 Homologues in Rabbits Does Not Impair Ixodes ricinus Feeding or Oviposition. PLoS ONE 10, e0123495.
Dai, J., Narasimhan, S., Zhang, L., Liu, L., Wang, P., Fikrig, E., 2010. Tick histamine release factor is critical for Ixodes scapularis engorgement and transmission of the lyme disease agent. PLoS Pathog. 6, e1001205.
Daix, V., Schroeder, H., Praet, N., Georgin, J.-P., Chiappino, I., Gillet, L., de Fays, K., Decrem, Y., Leboulle, G., Godfroid, E., Bollen, A., Pastoret, P.-P., Gem, L., Sharp, P. M., Vanderplasschen, A., 2007. Ixodes ticks belonging to the Ixodes ricinus complex encode a family of anticomplement proteins. Insect Mol. Biol. 16, 155-166.
de la Fuente, J., Moreno-Cid, J. A., Galindo, R. C., Almazan, C., Kocan, K. M., Merino, O., Perez de la Lastra, J. M., Estrada-Pena, A., Blouin, E. F., 2013. Subolesin/Akirin vaccines for the control of arthropod vectors and vectorborne pathogens. Transbound Emerg Dis 60 Suppl 2, 172-178.
Decrem, Y., Mariller, M., Lahaye, K., Blasioli, V., Beaufays, J., Zouaoui Boudjeltia, K., Vanhaeverbeek, M., Cerutti, M., Brossard, M., Vanhamme, L., Godfroid, E., 2008. The impact of gene knock-down and vaccination against salivary metalloproteases on blood feeding and egg laying by Ixodes ricinus. Int. J. Parasitol. 38, 549-560.
Eisen, L., 2018. Pathogen transmission in relation to duration of attachment by Ixodes scapularis ticks. Ticks and Tick-borne Diseases 9, 535-542.
Francischetti, I. M. B., Mather, T. N., Ribeiro, J. M. C., 2003. Cloning of a salivary gland metalloprotease and characterization of gelatinase and fibrin(ogen)lytic activities in the saliva of the Lyme disease tick vector Ixodes scapularis. Biochem. Biophys. Res. Commun. 305, 869-875.
Frimmel, S., Krienke, A., Riebold, D., Loebermann, M., Littmann, M., Fiedler, K., Klaus, C., Suss, J., Reisinger, E. C., 2014. Tick-borne encephalitis virus habitats in North East Germany: reemergence of TBEV in ticks after 15 years of inactivity. Biomed Res Int 2014, 308371.
Ginsberg, H. S., Albert, M., Acevedo, L., Dyer, M. C., Arsnoe, I. M., Tsao, J. I.,
Mather, T. N., LeBrun, R. A., 2017. Environmental Factors Affecting Survival of Immature Ixodes scapularis and Implications for Geographical Distribution of Lyme Disease: The Climate/Behavior Hypothesis. PLoS ONE 12, e0168723.
Gomes, H., Moraes, J., Githaka, N., Martins, R., Isezaki, M., Vaz, I. da S., Logullo, C., Konnai, S., Ohashi, K., 2015. Vaccination with cyclin-dependent kinase tick antigen confers protection against Ixodes infestation. Vet. Parasitol. 211, 266-273.
Hofmann, H., Fingerle, V., Hunfeld, K.-P., Huppertz, H.-I., Krause, A., Rauer, S., Ruf, B., Consensus group, 2017. Cutaneous Lyme borreliosis: Guideline of the German Dermatology Society. Ger Med Sci 15, Doc14.
Hust, M., Meysing, M., Schirrmann, T., Selke, M., Meens, J., Gerlach, G.-F., Dubel, S., 2006. Enrichment of open reading frames presented on bacteriophage M13 using hyperphage. Biotechniques 41, 335-42.
Ismail, N., McBride, J. W., 2017. Tick-Borne Emerging Infections: Ehrlichiosis and Anaplasmosis. Clin. Lab. Med. 37, 317-340.
John, M., Raman, M., Ryan, K., 2017. A tiny tick can cause a big health problem. Indian J Ophthalmol 65, 1228-1232.
Jonsson, N. N., Matschoss, A. L., Pepper, P., Green, P. E., Albrecht, M. S., Hungerford, J., Ansell, J., 2000. Evaluation of tickGARD(PLUS), a novel vaccine against Boophilus microplus, in lactating Holstein-Friesian cows. Vet. Parasitol. 88, 275-285.
Kotsyfakis, M., Anderson, J. M., Andersen, J. F., Calvo, E., Francischetti, I. M. B., Mather, T. N., Valenzuela, J. G., Ribeiro, J. M. C., 2008. Cutting edge: Immunity against a “silent” salivary antigen of the Lyme vector Ixodes scapularis impairs its ability to feed. J. Immunol. 181, 5209-5212.
Kotsyfakis, M., Karim, S., Andersen, J. F., Mather, T. N., Ribeiro, J. M. C., 2007. Selective cysteine protease inhibition contributes to blood-feeding success of the tick Ixodes scapularis. J. Biol. Chem. 282, 29256-29263.
Kügler, J., Nieswandt, S., Gerlach, G. F., Meens, J., Schirrmann, T., Hust, M., 2008. Identification of immunogenic polypeptides from a Mycoplasma hyopneumoniae genome library by phage display. Appl. Microbiol. Biotechnol 80, 447-58.
Meyer, T., Schirrmann, T., Frenzel, A., Miethe, S., Stratmann-Selke, J., Gerlach, G. F., Strutzberg-Minder, K., Dubel, S., Hust, M., 2012. Identification of immunogenic proteins and generation of antibodies against Salmonella Typhimurium using phage display. BMC Biotechnol. 12, 29.
Miller, R., Estrada-Pena, A., Almazan, C., Allen, A., Jory, L., Yeater, K., Messenger, M., Ellis, D., Perez de Leon, A. A., 2012. Exploring the use of an anti-tick vaccine as a tool for the integrated eradication of the cattle fever tick, Rhipicephalus (Boophilus) annulatus. Vaccine 30, 5682-5687.
Naseem, S., Meens, J., Jores, J., Heller, M., Dubel, S., Hust, M., Gerlach, G.-F., 2010. Phage display-based identification and potential diagnostic application of novel antigens from Mycoplasma mycoides subsp. mycoides small colony type. Vet. Microbiol 142, 285-292.
Nazario, S., Das, S., de Silva, A. M., Deponte, K., Marcantonio, N., Anderson, J. F., Fish, D., Fikrig, E., Kantor, F. S., 1998. Prevention of Borrelia burgdorferi transmission in guinea pigs by tick immunity. Am. J. Trop. Med. Hyg. 58, 780-785.
Nelson, C. A., Saha, S., Kugeler, K. J., Delorey, M. J., Shankar, M. B., Hinckley, A. F., Mead, P. S., 2015. Incidence of Clinician-Diagnosed Lyme Disease, United States, 2005-2010. Emerging Infect. Dis. 21, 1625-1631.
Ribeiro, J. M. C., Alarcon-Chaidez, F., Francischetti, I. M. B., Mans, B. J., Mather, T. N., Valenzuela, J. G., Wikel, S. K., 2006. An annotated catalog of salivary gland transcripts from Ixodes scapularis ticks. Insect Biochem. Mol. Biol. 36, 111-129.
Schetters, T., Bishop, R., Crampton, M., Kopaček, P., Lew-Tabor, A., Maritz-Olivier, C., Miller, R., Mosqueda, J., Patarroyo, J., Rodriguez-Valle, M., Scoles, G. A., de la Fuente, J., 2016. Cattle tick vaccine researchers join forces in CATVAC. Parasit Vectors 9, 105.
Schuijt, T. J., Narasimhan, S., Daffre, S., DePonte, K., Hovius, J. W. R., Van't Veer, C., van der Poll, T., Bakhtiari, K., Meijers, J. C. M., Boder, E. T., van Dam, A. P.,
Fikrig, E., 2011. Identification and characterization of Ixodes scapularis antigens that elicit tick immunity using yeast surface display. PLoS ONE 6, e15926. Shapiro, E. D., 2014. Borrelia burgdorferi (Lyme disease). Pediatr Rev 35, 500-509.
Shattuck, W. M. C., Dyer, M. C., Desrosiers, J., Fast, L. D., Terry, F. E., Martin, W. D.,
Moise, L., De Groot, A. S., Mather, T. N., 2014. Partial pathogen protection by tick-bite sensitization and epitope recognition in peptide-immunized HLA DR3 transgenic mice. Hum Vaccin Immunother 10, 3048-3059.
Trager, W., 1939. Accquired immunity to ticks. J Parasitol 25, 57-81. Valenzuela, J. G., Charlab, R., Mather, T. N., Ribeiro, J. M., 2000. Purification, cloning, and expression of a novel salivary anticomplement protein from the tick, Ixodes scapularis. J. Biol. Chem. 275, 18717-18723. https://doi.org/10.1074/jbc.M001486200
Valenzuela, J. G., Charlab, R., Mather, T. N., Ribeiro, J. M., 2000. Purification, cloning, and expression of a novel salivary anticomplement protein from the tick, Ixodes scapularis. J. Biol. Chem. 275, 18717-18723. https://doi.org/10.1074/jbc.M001486200
Vonesch, N., D'Ovidio, M. C., Melis, P., Remoli, M. E., Ciufolini, M. G., Tomao, P., 2016. Climate change, vector-borne diseases and working population. Ann. 1st. Super. Sanita 52, 397-405.
Wikel, S. K., 1996. Host immunity to ticks. Annu. Rev. Entomol. 41, 1-22.
Willyard, C., 2014. Resurrecting the “yuppie vaccine.” Nat. Med. 20, 698-701.
Zantow, J., Dubel, S., Hust, M., 2016. Funktionales Proteom-Display zur Identifikation von Biomarkern. Biospektrum 22, 256-259.
Zantow, J., Moreira, G. M. S. G., Dubel, S., Hust, M., 2018. ORFeome Phage Display. Methods Mol. Biol. 1701, 477-495.

1. A combination comprising at least the following components:
(a) (i) a metalloprotease 1 from a tick species particularly selected from I. ricinus, I. persulcatus, I. scapularis, and I. pacificus,
(ii) a polypeptide having a sequence identity of at least 80%, 85%, 90%, 95% or 98% to a polypeptide according to (i),
(iii) an immunogenic fragment of a polypeptide according to (i) or (ii) comprising at least 6, at least 8, at least 10, at least 12 or at least 15 contiguous amino acids of a polypeptide according to (i) or (ii),
(iv) a nucleic acid molecule encoding a polypeptide according to (i), (ii) or (iii), and
(b) (i) a thrombin inhibitor from a tick species particularly selected from I. ricinus, I. persulcatus, I. scapularis, and I. pacificus,
(ii) a polypeptide having a sequence identity of at least 80%, 85%, 90%, 95% or 98% of a protein according to (i),
(iii) an immunogenic fragment of a polypeptide according to (i) or (ii) comprising at least 6, at least 8, at least 10, at least 12 or at least 15 contiguous amino acids of a polypeptide according to (i) or (ii), or
(iv) a nucleic acid molecule encoding a polypeptide according to (i), (ii) or (iii).

2. The combination of claim 1 comprising at least one further component selected from
(c) (i) an immunogenic polypeptide from a tick species selected from I. ricinus, I. persulcatus, I. scapularis, and I. pacificus,
(ii) a polypeptide having a sequence identity of at least 80%, 85%, 90% or 95% of a protein according to (i),
(iii) an immunogenic fragment of a polypeptide according to (i) or (ii) comprising at least 6, at least 8, at least 10, at least 12 or at least 15 contiguous amino acids of a polypeptide according to (i) or (ii), or
(iv) a nucleic acid molecule encoding a polypeptide according to (i), (ii) or (iii).

3. The combination of claim 1, wherein component (a) is a metalloprotease 1 from I. scapularis, as shown in SEQ ID NO: 1 or an immunogenic homologue or an immunogenic fragment thereof or a nucleic acid molecule coding therefor.

4. The combination of claim 1, wherein component (b) is a thrombin inhibitor from I. scapularis, as shown in SEQ ID NO: 2 or an immunogenic homologue or an immunogenic fragment thereof or a nuclei acid molecule coding therefor.

5. The combination of claim 1 which is a composition comprising several components in a single container or a kit comprising individual components in separate containers.

6. The combination of claim 1 for use in medicine.

7. The combination of claim 1 for use in human medicine.

8. The combination of claim 1 for use as a tick vaccine, particularly a human tick vaccine.

9. The combination of claim 1 for use as a vaccine against ticks of the genus Ixodes.

10. The combination of claim 1 for use as a vaccine, against several different tick species of the genus Ixodes.

11. The combination of claim 1 further comprising an adjuvant.

12. The combination of claim 1 for use in preventing a tick-borne pathogen infection.

13. The combination of claim 1 for use in preventing an infection by a pathogen selected from Babesia spec., Borrelia spec., Francisella spec., Rickettsia spec., Anaplasma spec., Ehrlichia spec., tick-borne encephalitis virus (TBEV), Powassan virus, Colorado tick fever virus, Crimean-Congo hemorrhagic fever virus or Heartland virus.
